(19) United States
(12) Patent Application Publication
(10) Pub. No.: US 2025/0339047 A1
(43) Pub. Date: Nov. 6, 2025

(54) Non-Invasive Intracranial Pressure Sensing System and Method

(71) Applicant: CoMind Technologies Limited, London (GB)

(72) Inventors: William PARKER, London (GB); Pablo Ortega SAN MIGUEL, London (GB); Matthew VALLEY, London (GB); David BORYCKI, London (GB); Alexander ANTROBUS, London (GB); Alexandra TRAN VAN MINH, London (GB); Rory BEARD, London (GB)

(21) Appl. No.: 18/868,533

(22) PCT Filed: May 26, 2023
(86) PCT No.: PCT/GB2023/051394

§ 371 Date: Nov. 22, 2024

(30) Foreign Application Priority Data
May 26, 2022 (GB) 2207775.4

(51) Int. Cl. A61B 5/03; A61B 5/00; A61B 5/021; A61B 5/026

(52) U.S. Cl. CPC A61B 5/031; A61B 5/02108; A61B 5/0261; A61B 5/6814; A61B 2562/04

(57) ABSTRACT

An aspect of the disclosure provides a non-invasive intracranial pressure sensing apparatus comprising an interferometric near infrared spectroscopy, iNIRS system, the iNIRS system comprising: a light emitting arrangement comprising: a light source configured to emit light; a sample delivery channel coupled to the light source and arranged to be coupled to the subject's scalp to direct light from the light source towards the subject's brain tissue; and a reference channel coupled to the light source for receiving light therefrom; a light detecting arrangement configured to be coupled to the subject's scalp and the light emitting arrangement, the light detecting arrangement comprising an interferometric optical detector configured to receive: (i) reference light from the reference channel, and (ii) sample light from the subject's brain, the sample light comprising light emitted from the light source; wherein the optical detector is arranged to combine the sample light with the reference light to provide combined light signals comprising one or more components at a beat frequency between sample light and reference light; and wherein the sensing apparatus comprises a controller configured to process data indicative of the combined light signals to determine an indication of intracranial blood pressure for the subject based on at least one property of a pulsatile waveform of one or more identified pulses of blood flow through the subject's brain.

### TECHNICAL FIELD

The present disclosure relates to the field of neuroimaging and analysis. In particular, the present disclosure relates to interferometric near infrared spectroscopy (‘iNIRS’) systems and methods for neuroimaging and analysis.

### BACKGROUND

Near infrared spectroscopy (‘NIRS’) is a spectroscopic method which uses the near infrared region of the electromagnetic spectrum (e.g. between 780 and 2500 nm). NIRS systems can be used to provide non-invasive monitoring of scattering and absorption properties of a medium. Radiation at NIRS wavelengths is less easily absorbed by human skin (and also bones) than visible light, and so NIRS radiation may penetrate both skin and skull, and penetrate into brain tissue. NIRS may be used as a technique for non-invasive imaging of human brain tissue by monitoring scattering and absorption properties of the NIRS radiation within the brain tissue.

While NIRS methods can be extended for monitoring oxygenation, when multiple wavelengths are used, the blood flow monitoring is necessary to infer information about metabolism. Diffuse correlation spectroscopy (DCS) can be used to noninvasively monitor blood flow in the brain by measuring temporal fluctuations of the light remitted from the sample. DCS can further extract other brain metrics, including intracranial pressure (ICP). However, to extract ICP existing DCS approaches require additional devices, and heavy averaging, making the final approach bulky and slow. Also, to quantify the blood flow DCS requires optical properties, which usually are assumed or achieved from the separate NIRS instrument. Finally, since DCS and NIRS relies only on the light intensities, they reject half of the information about the scattered light, encoded in the optical phase. Consequently, measurements are affected by additional assumptions from disregarding phase information.

It is thus desirable to provide improved technology for neuromonitoring and analysis, which will combine NIRS and DCS into a single modality, and rapidly provide optical and dynamical properties of the biological tissues.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided a non-invasive intracranial pressure sensing apparatus comprising an interferometric near infrared spectroscopy (‘INIRS’) system. The iNIRS system comprises: a light emitting arrangement comprising: a light source configured to provide (e.g. wavelength swept) emission of light (e.g. a light source configured to emit light with a swept wavelength); a sample delivery channel coupled to the light source and arranged to be coupled to the subject's scalp to direct light from the light source towards the subject's brain tissue; and a reference channel coupled to the light source for receiving light therefrom; a light detecting arrangement configured to be coupled to the subject's scalp and the light emitting arrangement, a light detecting arrangement comprising an interferometric optical detector configured to receive: (i) reference light from the reference channel, and (ii) sample light from the subject's brain tissue, the sample light comprising light emitted from the light source (e.g. some of which may have travelled from the light source through the subject's scalp and skull and through a portion of their brain tissue before scattering towards the detector). The optical detector is arranged to combine the sample light with the reference light to provide combined light signals comprising one or more components at a beat frequency between sample light and reference light. The sensing apparatus comprises a controller configured to process data indicative of the combined light signals to determine an indication of intracranial blood pressure for the subject based on at least one property of a pulsatile waveform of one or more identified pulses of blood flow through the subject's brain tissue.

Embodiments may enable intracranial blood pressure (‘ICP’) to be obtained non-invasively. This may be advantageous, as alternative approaches for determining ICP can be very invasive, to such an extent that their use may be limited to only situations where a measurement of ICP is most necessary (and thus warrants the high risk associated with invasively measuring ICP).

Processing the data indicative of the combined light signals may comprise obtaining blood flow data indicative of one or more pulses of blood flow through the subject's brain. For example, this may comprise data containing values for a cerebral blood flow index. The cerebral blood flow index values may provide an indication of velocity for movement of blood in a given volume of the subject's brain. The controller may be configured to obtain extracerebral blood flow data indicative of one or more pulses of blood flow through an extracerebral region of the subject's body. The extracerebral blood flow data may contain blood pressure values for one or more pulses of extracerebral blood flow. The extracerebral blood flow data may contain blood flow index values for one or more pulses of extracerebral blood flow (and the controller may be configured to determine corresponding extracerebral blood pressure values therefrom).

The controller may be configured to determine the indication of intracranial blood pressure for the subject based on both the cerebral blood flow data and the extracerebral blood flow data. The controller may determine the indication of ICP based on a comparison between the cerebral blood flow data and the extracerebral blood flow data. Comparing the two may comprise aligning cerebral blood flow data with the extracerebral blood flow data so that pulses of blood through the subject's brain tissue are aligned with corresponding pulses of blood flow through the extracerebral region of the subject's body. Aligning may comprise associating each cerebral pulse of blood flow with a corresponding extracerebral pulse of blood flow (e.g. so that the two pulses correspond to the same heartbeat cycle-even if they were slightly out of phase with each other due to being measured in different regions of the subject's body). Comparing two pulses may comprise comparing: (i) one or more properties of the pulsatile waveform for a pulse of blood flow through the subject's brain tissue, and (ii) one or more properties of the pulsatile waveform for a pulse of blood flow through the extracerebral region of the subject's body.

The extracerebral blood flow data may contain an indication of blood pressure values for the pulses of blood flow through the extracerebral region of the subject's body. For example, the extracerebral blood flow may be for a superficial (e.g. less deep) region of the subject's body, such as their scalp (above their skull). For example, the extracerebral blood flow data may comprise a series of values for the subject's blood pressure in the extracerebral region of their body (e.g. a time-ordered series). The extracerebral blood flow data may provide blood pressure values for the pulsatile waveform for each pulse of blood flow (e.g. which indicates values for blood pressure at a plurality of points during each pulse of blood flow through the extracerebral region of the subject's body). The controller may be configured to determine the indication of intracranial blood pressure for the subject based on: (i) the pulsatile waveform of one or more identified pulses of blood flow through the subject's brain tissue, and (ii) a corresponding pulsatile waveform for the blood pressure for one or more pulses of blood flow through the extracerebral region of the subject's body.

For example, the controller may be configured to determine the ICP based on an extracerebral blood pressure value at a given point during the extracerebral blood pressure pulsatile waveform (e.g. where that given point during the pulsatile waveform corresponds to a selected point during the pulsatile waveform for cerebral blood flow, such as a minima for cerebral blood flow values). The controller may be configured to determine the indication of intracranial blood pressure for the subject based on an identified critical closing pressure for a blood vessel in the subject's brain tissue. The critical closing pressure may comprise the value for pressure at which the blood vessel in the subject's brain tissue closes. For example, the controller may be configured to detect the blood vessel closing based on a change in the cerebral blood flow data (e.g. the cerebral blood flow value dropping, such as to a zero value or a known minimum value). The controller may be configured to identify the point during the cerebral pulsatile waveform at which the blood vessel closes. The controller may be configured to identify a corresponding point during the pulsatile waveform for extracerebral pressure. The controller may be configured to determine the ICP based on a corresponding pressure being attributed to the blood vessel (and with the blood vessel closing due to surrounding ICP).

The controller may be configured to process the data indicative of the combined light signals to obtain cerebral blood flow index data for blood flow through the subject's brain tissue. The pulsatile waveform of one or more identified pulses of blood flow through the subject's brain tissue may comprise a pulsatile waveform for the cerebral blood flow index. For example, the cerebral blood flow data may comprise a series of values for cerebral blood flow index (‘CBFi’), e.g. a time-ordered series of cerebral blood flow index values. The controller may be configured to determine ICP based on one or more properties associated with these CBFi values. For example, the property may relate to a shape of the pulsatile waveform, e.g. a property indicative of the shape of each pulse. The property may comprise an indication of at least one of: maximum and/or minimum values, a difference between maximum and minimum values, an average and/or a variance for the values, a rate of change of values etc.

The sensing apparatus may be configured to obtain the extracerebral blood flow data using the iNIRS system. The iNIRS system may be configured to obtain both the extracerebral blood flow data and the cerebral blood flow data using the same source-detector channel. The iNIRS system may be configured to obtain extracerebral blood flow index data. The controller may be configured to process the extracerebral blood flow index data to obtain values for extracerebral blood pressure. The light detecting arrangement may be configured to be coupled to the subject's scalp for the detector to obtain combined light signals comprising both: (i) components at beat frequencies associated with sample light travelling from the subject's brain tissue, and (ii) components at beat frequencies associated with sample light travelling from the extracerebral region of the subject's body. The controller may be configured to demix data associated with the subject's brain tissue from data associated with the extracerebral region of the subject's body (e.g. to separate out data from the same combined light signals into two separate data groups: one for cerebral data, and one for extracerebral data). The controller may be configured to demix the data based on time of flight for the sample light. The controller may be configured to determine the cerebral blood flow data and the extracerebral blood flow data based on the demixed data.

The iNIRS system may comprise a plurality of optical detectors. Each optical detector may be configured to obtain cerebral blood flow data for the subject's brain tissue. The controller may be configured to determine the indication of intracranial blood pressure based on properties of pulsatile waveforms of pulses of blood flow through the subject's brain tissue detected by the plurality of detectors. The apparatus may be configured to obtain extracerebral blood flow data for different extracerebral regions of the subject's body based on combined light signals associated with the different detectors. The detectors may be arranged to be spatially distributed about the subject's scalp so that at least some of the pulses of blood flow detected by the different detectors are from different portions of the subject's brain tissue. The iNIRS system may comprise two source-detector channels: (i) a cerebral source-detector channel configured to obtain cerebral blood flow data, and (ii) an extracerebral source-detector channel configured to obtain extracerebral blood flow data. The sensing apparatus may comprise an extracerebral blood flow sensor configured to obtain extracerebral blood flow data. The extracerebral blood flow sensor may be configured to obtain pressure values for pulses of extracerebral blood flow.

The controller may be configured to determine the indication of intracranial blood pressure based on a difference between diastolic and systolic values for the one or more pulses of blood flow through the subject's brain tissue. The controller may be configured to determine the indication of intracranial blood pressure based on a pulsatility index for the pulses of blood flow through the subject's brain tissue. The controller may be configured to determine the indication of intracranial blood pressure for the subject based on a difference in shape between: (i) one or more pulses of blood flow through the subject's brain tissue, and (ii) one or more pulses of blood flow through the extracerebral region of the subject's body. The controller may be configured to determine the indication of intracranial blood pressure for the subject based on diastolic values for the pulses of blood flow. The iNIRS system may comprise two or more light sources, wherein a first of the light sources is configured to provide (e.g. wavelength swept) emission of light through a plurality of wavelengths above an oximetry isosbestic wavelength, and wherein a second of the light sources is configured to provide (e.g. wavelength swept) emission of light through a plurality of wavelengths below an oximetry isosbestic wavelength. The controller may be configured to determine the indication of intracranial blood pressure based on sample light received from each of the two light sources.

The controller may be configured to obtain time of flight data based on the combined light signals, wherein the time of flight data comprises a data surface containing a time-ordered series of time of flight distributions for photons of sample light reaching the optical detector from the light source. The controller may be configured to determine cerebral blood flow data based on changes in the data surface. The controller may be configured to determine the cerebral blood flow data based on a decay rate associated with the data surface. The controller may be configured to obtain an indication of one or more optical properties of the subject's brain tissue based on the time of flight data. The one or more optical properties of the subject's brain tissue may comprise scattering and/or absorption coefficients. The controller may be configured to determine the cerebral blood flow data for the subject's brain tissue based on: (i) the one or more optical properties of the subject's brain tissue, and (ii) changes in intensity of the sample light received at the optical detector.

In an aspect, there is provided a method of non-invasive intracranial pressure sensing, the method comprising: operating a light source to provide (e.g. wavelength swept) emission of light; delivering light from the light source through both: (i) a sample channel and towards the subject's brain tissue, and (ii) a reference channel; receiving at an interferometric optical detector: (i) reference light from the reference channel, and (ii) sample light from the subject's brain tissue, the sample light comprising light emitted from the light source; combining, at the optical detector, the sample light with the reference light to provide combined light signals comprising one or more components at a beat frequency between sample light and reference light; processing data indicative of the combined light signals to determine an indication of intracranial blood pressure for the subject based on at least one property of a pulsatile waveform of one or more identified pulses of blood flow through the subject's brain tissue.

In an aspect, there is provided a non-invasive intracranial pressure sensing apparatus comprising an interferometric near infrared spectroscopy, iNIRS, system, the iNIRS system comprising: a light emitting arrangement comprising: a light source configured to provide (e.g. wavelength swept) emission of coherent light; a sample delivery channel coupled to the light source and arranged to be coupled to the subject's scalp to direct light from the light source towards the subject's brain tissue; and a reference channel coupled to the light source for receiving light therefrom; a light detecting arrangement configured to be coupled to the subject's scalp and the light emitting arrangement, the light detecting arrangement comprising: an interferometric optical detector configured to receive: (i) reference light from the reference channel, and (ii) sample light from the subject's brain tissue, the sample light comprising light emitted from the light source, wherein the optical detector is arranged to combine the sample light with the reference light to provide combined light signals comprising one or more components at a beat frequency between sample light and reference light; and signal processing and conversion circuitry coupled to the detector and configured to produce combined light signal data from the combined light signals; wherein the sensing apparatus comprises a controller configured to: process the combined light signal data to obtain time of flight data comprising a plurality of time of flight distributions for photons of sample light reaching the optical detector from the light source; process the time of flight data to obtain an indication of one or more optical properties of the subject's brain tissue; determine cerebral blood flow data for the subject's brain tissue based on: (i) the one or more optical properties of the subject's brain tissue, and (ii) changes in intensity of the sample light received at the optical detector, wherein the cerebral blood flow data contains an indication of one or more pulses of blood flow through the subject's brain tissue; and determine an indication of intracranial blood pressure for the subject based on a shape of the one or more pulses of blood flow through the subject's brain tissue.

Aspects of the present disclosure include one or more computer program products comprising computer program instructions to program a processor to control operation of an interferometric near infrared spectroscopy system, e.g. to control operation of a light emitting arrangement and a light detecting arrangement, to perform any methods disclosed herein.

Embodiments may provide iNIRS systems and methods for neuroimaging and analysis of a subject's brain tissue. The iNIRS systems and methods of the present disclosure are directed to a fundamentally different approach for performing neuroimaging and analysis, as compared to the fNIRS technologies described above. Embodiments may provide an improved approach for performing iNIRS neuroimaging and analysis. As disclosed herein, iNIRS systems of the present disclosure include two light sources and one or more light detectors. The iNIRS systems of the present disclosure may also include a controller arranged to receive output signals from the one or more light detectors.

Each light source may comprise a light generating element arranged to generate light (e.g. near infrared light). For example, each light generating element may comprise a laser. Each light source may comprise an optical arrangement coupled to the light generating element. The optical arrangement of each light source may be configured to deliver the generated light from the light generating element to each of one or more different locations. The optical arrangement of each light source may be arranged to direct some of the light from the light generating element towards a region to be sampled. The optical arrangement of each light source may be arranged to direct some of the light to each light detector. The optical arrangement of each light source may comprise a plurality of light delivery channels. The plurality of light delivery channels may include one or more sample delivery channels, and/or one or more reference delivery channels. Each light delivery channel may comprise an optical channel, such as an optical fibre. Each light delivery channel may be configured for transmitting light along its length (e.g. from the light generating element towards the subject's scalp or the light detector). The optical arrangement of each light source may comprise a light splitter for splitting light into each of the different delivery light channels.

The iNIRS system may be arranged so that, when installed on a subject's head (e.g. for providing neuroimaging and analysis of that subject's brain tissue), the optical arrangement of each light source is configured to direct some of the light towards the subject's scalp. For example, the optical arrangement of each light source may comprise a sample delivery channel (e.g. which is operable for directing sample light towards the subject's scalp). The iNIRS system may be arranged so that, in use, the optical arrangement of each light source may direct some of the light directly to the light detectors (e.g. for combining with sample light from the subject's brain tissue). For example, the optical arrangement of each light source may comprise a reference delivery channel (e.g. which is operable for directing reference light to one or more of the light detectors). The optical arrangement of each light source may be configured to deliver light from the light generating element to each light channel. The optical arrangement of each light source may be configured to deliver both: (i) light to the sample light delivery channel (‘sample light’), and (ii) light to the reference delivery channel (‘reference light’). For example, the optical arrangement of each light source may comprise a light splitter configured to split light received from the light generating element into each of the different channels.

The iNIRS system may be arranged so that, in use when installed on a subject's brain tissue, the sample light may be directed towards the subject's scalp and brain tissue (e.g. through the sample delivery channel), and the reference light may be directed towards each light detector (e.g. through the reference delivery channel). Each light source may be arranged to provide (e.g. wavelength swept) emission of light (e.g. each light source may be arranged to output light at each of a plurality of different wavelengths in a selected time period). For example, each light source may comprise a modifying element for controlling operation of the light generating element to output light at each of a plurality of different wavelengths. Each light source may be arranged to sweep the wavelength of the light it outputs (e.g. increasing or decreasing in wavelength). Each light source may be arranged to provide chirped emission of light in which, each chirp (or ‘pulse’) comprises one wavelength sweep. Each light source may be arranged to output sequential chirps with the same wavelength sweep, e.g. such that the wavelength of the light output from the light source changes according to a repeating pattern.

Each light detector may provide an interferometric optical detector. Each light detector may comprise an optical arrangement. The optical arrangement of the light detector is configured to direct light to be detected into the light detector (e.g. from the subject's scalp). The optical arrangement of the light detector may comprise a plurality of light receiving channels. The plurality of light receiving channels may include one or more sample light receiving channels, and/or one or more reference light receiving channels. Each light receiving channel may comprise an optical channel, such as an optical fibre.

The iNIRS system may be arranged so that, when installed on a subject's head (e.g. for providing neuroimaging and analysis of that subject's brain tissue), the optical arrangement of the light detector is configured to receive light emitted from the light source (e.g. which has travelled through the subject's brain tissue from the light source). For example, the optical arrangement of the light detector may comprise a sample receiving channel (e.g. which is operable for receiving sample light from each light source which has passed through the subject's brain tissue). The iNIRS system may be arranged so that, in use, the optical arrangement of the light detector may receive some of the light from each light source which has travelled directly from the light source (e.g. which has travelled along an optical channel). For example, the optical arrangement of the light detector may comprise a reference receiving channel (e.g. which is operable for receiving reference light from one or more light sources). Each light detector may be coupled to each light source so that the reference delivery channel of the light source is coupled to the reference receiving channel of the light detector (e.g. so that reference light may travel from the light generating element to the light detector via the reference delivery and receiving channels).

The optical arrangement of the light detector may be configured to deliver both to the light detector: (i) light from the sample light receiving channel (‘sample light’), and (ii) light from the reference receiving channel (‘reference light’). For example, in use, the detector is arranged to receive both: (i) sample light from each light source which has passed through the subject's brain tissue, and (ii) reference light from each light source which has travelled to the light detector along one or more reference channels.

The light detector may be arranged to combine reference light with sample light to provide a combined light signal. For example, the light detector may comprise a light combiner (e.g. for combining light on the reference receiving channel with light on the sample receiving channel). The combined light signal may include a plurality of components at beat frequencies, e.g. at frequencies corresponding to the differences in wavelength between the sample light and the reference light. Each light detector is configured to convert received combined light signals into one or more electrical signals indicative of that combined light signal. For example, the detector may comprise one or more photodiodes. Each photodiode may output an electrical signal (e.g. a current) indicative of the combined light signal. The detector may comprise a balanced photodetector (e.g. which includes two photodiodes, which may be 180° out of phase with each other, and its output may be a combination of the two photodiode current outputs). The detector may optionally include current to voltage conversion circuitry and/or one or more amplifiers for amplifying the electrical signal.

The iNIRS system may include at least one analogue to digital converter arranged to convert electrical signals representing the sample light (e.g. the combined light signals) into one or more digital signals. The controller is arranged to process the digital signals to determine one or more properties of the subject's brain tissue. The controller may be configured to determine optical properties of the subject's brain tissue (e.g. for absorption and/or scattering). The controller may be configured to determine one or more dynamic properties of the subject's brain tissue (e.g. properties of the subject's brain tissue which are varying over time). For example, the controller may be configured to detect the presence of movement within the subject's brain tissue (e.g. due to movement, such as flow, of blood within the brain tissue).

The controller may be configured to process the digital signals to obtain time of flight information for photons of sample light travelling from each light source through the subject's brain tissue to the light detector. The controller may be configured to identify penetration depths (and optionally expected trajectories for photons through the brain tissue) associated with the different times of flight for sample light photons. The controller may be configured to obtain a time-ordered series of time of flight distributions for sample light photons reaching each light detector. The controller may be configured to process the time-ordered series to identify changes in the time of flight distribution over time, such as identifying decay and/or decay rates between success time of flight distributions. The controller may be configured to provide depth-resolved processing, e.g. by filtering the time of flight data to focus on only photons within a selected time of flight range (e.g. to identify changes in optical properties of the brain tissue for penetration depth(s) associated with that time of flight range). The controller may be configured to process data received from the light detector(s) to provide time of flight information with depth-resolved autocorrelations for the subject's brain tissue.

The controller may be configured to process the received data indicative of sample light received at a light detector and to output a control signal based on that received data. The control signal may provide an indication of the time of flight distribution (e.g. the controller may be configured to output the time of flight distribution). The control signal may provide an indication of one or more properties determined based on the time of flight distribution, such as optical properties for the brain tissue (e.g. scattering and/or absorption coefficients, and/or how these have changed/are changing). The control signal may provide an indication of blood flow within the subject's brain tissue. The control signal may provide a depth-resolved indication of one or more properties of the subject's brain tissue (e.g. linked to a specific region within their brain tissue, such as at a selected penetration depth range). The control signal may comprise an indication of one or more properties of the subject's brain tissue, such as intracranial pressure, blood flow index, artery elasticity, cerebral metabolic rate of oxygen consumption. The medical properties may be associated with specific regions/depths within the subject's brain tissue. The control signal may comprise an actuation command for a brain-computer interface, e.g. to control operation of a device based on the actuation command. The control signal may comprise an image for display, where that image represents a portion of the subject's brain tissue (as determined based on the received sample light).

### FIGURES

Some examples of the present disclosure will now be described, by way of example only, with reference to the figures, in which:

FIG. 1 shows a schematic diagram of an iNIRS system.

FIG. 2 shows a schematic diagram illustrating cerebral blood flow index data.

FIGS. 3a to 3c show schematic diagrams illustrating a method of processing data obtained using an iNIRS system.

FIG. 4a shows a schematic diagram of an iNIRS system.

FIG. 4b shows a schematic diagram illustrating a method of processing data obtained using an iNIRS system.

In the drawings like reference numerals are used to indicate like elements.

### SPECIFIC DESCRIPTION

The present disclosure relates to non-invasively monitoring intracranial pressure (‘ICP’). An interferometric near infrared spectroscopy (‘INIRS’) system is used to obtain time of flight data for photons of light travelling from a light source to a light detector (where at least some of those photons will have travelled through the subject's brain tissue). Based on a temporal evolution of this time of flight data, one or more properties associated with the flow of blood within the subject's brain tissue may be identified. In particular, a pulsatile waveform may be identified for a pulse of blood flow through the subject's brain tissue. An indication of ICP for the subject's brain may be determined based on one or more properties of this pulsatile waveform. To further improve ICP accuracy, one or more properties of extracerebral blood flow may also be obtained, and these properties may be compared with the cerebral blood flow properties. The iNIRS system may be configured to obtain both the cerebral blood flow data and the extracerebral blood flow data. An indication of ICP may be determined based on differences between pulsatile waveforms for the cerebral and extracerebral blood flow. Such differences may be indicative of constraining forces on the pulses of cerebral blood flow which are not present for the extracerebral blood flow (e.g. because the skull is of a fixed volume).

### Interferometric Near Infrared Spectroscopy (‘iNIRS’)

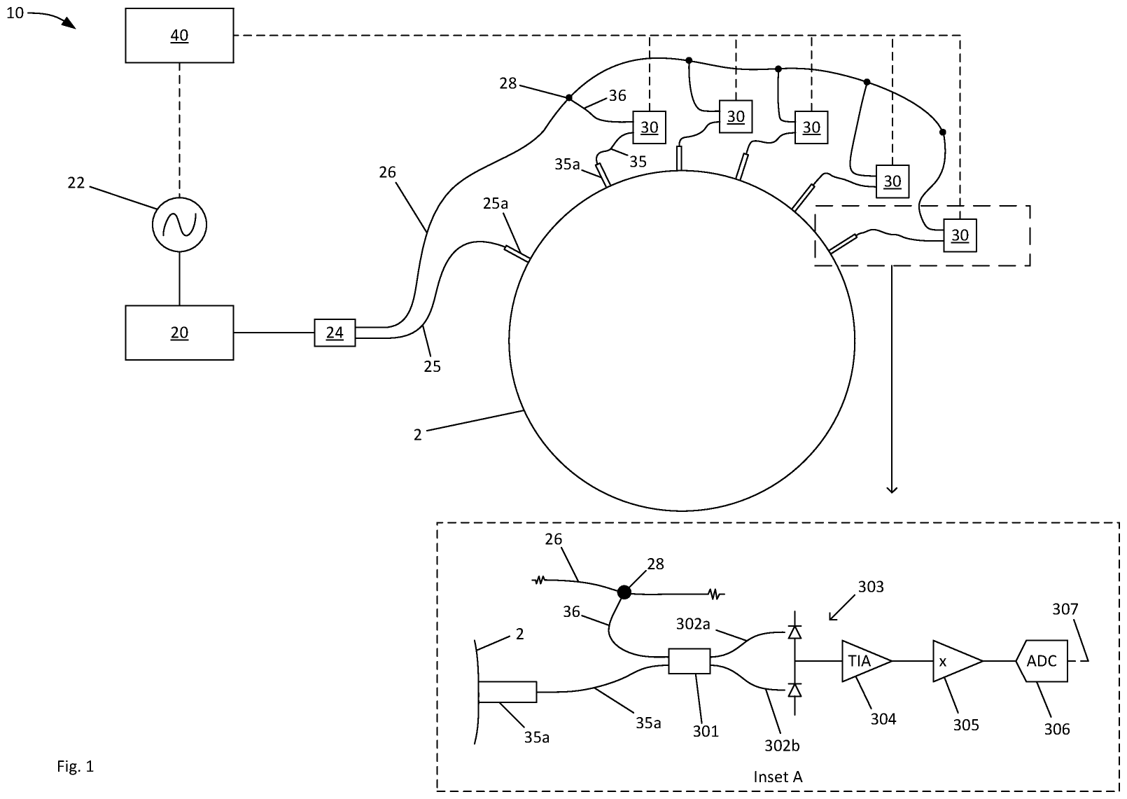

FIG. 1 shows a schematic diagram of an interferometric Near Infrared Spectroscopy (‘iNIRS’) system 10. The iNIRS system 10 includes a light source 20, a plurality of light detectors 30, and a controller 40. Inset A of FIG. 1 shows a more detailed view of one of the light detectors 30.

The iNIRS system 10 includes a light source modifier 22, and a light splitter 24. The iNIRS system 10 includes a sample delivery channel 25 and a reference delivery channel 26. The iNIRS system 10 is shown coupled to a subject's head 2. The iNIRS system 10 includes a sample delivery probe 25a and a plurality of sample receiving probes 35a. For each light detector 30, there is an associated sample receiving probe 35a, a sample receiving channel 35, a reference delivery channel connection 28, and a reference receiving channel 36.

The light source modifier 22 may comprise a source for providing a variable electrical control signal (e.g. a variable current or voltage provider). The light source modifier 22 is coupled to the light source 20. The light source modifier 22 may be electrically connected to the light source 20 to provide a variable current/voltage thereto.

The light source 20 may comprise a laser. For example, the laser may be a Distributed Feedback laser (‘DFB’) or a MEMS-Vertical Cavity Surface Emitting laser (‘MEMS-VCSEL’). The light source 20 is coupled to the light splitter 24. The light splitter 24 has an input for receiving light from the light source 20. The light splitter 24 has two outputs for transmitting light from the light source 20 to two separate channels. The sample delivery channel 25 is coupled to the light splitter 24 (to receive light therefrom), as is the reference delivery channel 26. The sample delivery channel 25 couples the light splitter 24 to the sample delivery probe 25a. The sample delivery probe 25a will be placed at a location on the subject's scalp.

Other types of suitable laser include a Distributed Bragg Reflector laser (‘DBR’), a Fourier Domain Mode Locking laser (‘FDML’), a Vertical Cavity Surface-Emitting laser (‘VCSEL’). Additionally, or alternatively, a pulsed supercontinuum laser may be used in combination with a pulse stretching mechanism, such as a grating or GRISM pulse stretcher or length of dispersive optical fibre. For example, such an arrangement may be configured to temporally separate the wavelengths in the pulse such that a frequency chirped pulse is created (e.g. for ultimately providing an interferogram when sample and reference pulses are compared).

The reference delivery channel 26 couples the light splitter 24 to each of the light detectors 30. For each detector, the reference delivery connection 28 couples the reference delivery channel 26 to the reference receiving channel 36 for that detector. Each reference receiving channel 36 is coupled to its light detector 30. Each light detector 30 is connected to the light source 20 to directly receive reference light therefrom (via one or more reference channels). Each sample receiving probe 35a is placed on the subject's scalp. Each sample receiving probe 35a is coupled to the sample receiving channel 35. Each sample receiving channel 35 is coupled to its light detector 30. Each light detector 30 is connected for indirectly receiving sample light from the light source 20 (via the sample delivery and receiving channels, and via the subject's brain tissue therebetween).

There are a plurality of different light detectors 30. Each detector may comprise an interferometer, such as a Mach-Zehnder interferometer. Each of the different light detectors 30 is coupled to the same light source 20 (each via one or more reference channels). The light detectors 30 may be spatially separated from the light source 20. The light detectors 30 may also be spatially separated from one another or they may be co-located on a sufficiently similar region of tissue that the received signals can be averaged together. For reference light to reach the light detector(s) 30 from the light source 20, the reference light will travel directly along one or more reference channels. For sample light to reach the light detector 30 from the light source 20, the sample light will travel indirectly via the subject's brain tissue. The sample light is delivered to the subject's scalp via one or more delivery channels. The sample light may then pass through the subject's brain tissue where it will be received and transmitted to a light detector 30 via one or the sample receiving channels 35. The illumination of the subject's brain tissue may thus occur using a different light channel to the detection of light from the subject's brain tissue.

The controller 40 may comprise any suitable component with data receiving and processing functionality. For example, the controller 40 may include at least one Application Specific Integrated Circuit (‘ASIC’). Other examples for the controller 40 may include a Field Programmable Gate Array (‘FPGA’) and/or a Data Acquisition module (‘DAQ’). The controller 40 is coupled to each of the detectors. The controller 40 may be connected to each detector via a wired connection (for receiving electrical signals indicative of detection therefrom), and/or the connection may be wireless (for receiving transmitted data indicative of detection therefrom). The controller 40 is coupled to the light source modifier 22. This connection may be wired or wireless.

The iNIRS system 10 may be at least partially housed within a garment for the subject's head 2. For example, the iNIRS system 10 may be provided in a hat/cap which is to be worn by the subject on their head 2. The head garment may be arranged to hold the light source 20 and detectors in a fixed arrangement relative to the subject's scalp. Some or all of the components may be provided with the head garment. For example, the head garment may include a plurality of receiving portions for receiving light source(s) 20 and light detectors 30. Channels connecting the light sources and light detectors 30 may be provided as part of the head garment (e.g. they may be routed through corresponding channel receiving portions of the head garment). The controller 40 may be separate to the head garment (e.g. and connected wirelessly) or it may also be provided as part of the head garment (e.g. by an ASIC within the head garment which may be wire coupled to the detectors and/or light source modifier 22). For example, the garment may be configured to receive the source and detection channels and the probes, with the other components of the system located elsewhere.

Some or all of the channels of the iNIRS system 10 may be provided by optical fibres. Light splitters of the present disclosure may comprise fibre-optic splitters. The iNIRS system 10 may include lenses, reflection and/or refraction devices for beam steering, as relevant. For example, the sample delivery probe 25a may include one or more lenses for spatially distributing sample light from the sample delivery channel 25 towards the subject's brain tissue. As another example, one or more of the sample receiving probes 35a may include a lens for focussing received light into the sample receiving channel 35 connected to that sample receiving probe 35a. As another example, the probes may be bare fibres which have been cleaved and/or polished.

The iNIRS system 10 is arranged to provide a plurality of source-detector pairs for each light source 20. In other words, the iNIRS system 10 is arranged so that each light detector 30 may receive two forms of light: (i) reference light, and (ii) sample light. Each detector is arranged to receive reference light directly from the light source 20 (the reference light will travel from the light source 20 along one or more channels to the light detector 30, e.g. without passing through the subject's brain tissue). Each detector is also arranged to receive sample indirectly from the light source 20 (the sample light will have been directed towards the subject's scalp tissue and a portion may have travelled through their brain tissue en route to the detector, e.g. the sample light will not have travelled exclusively through optical channels between the light source 20 and light detector 30).

The detectors 30 are arranged to be positioned on the subject's scalp to provide imaging of a selected region of their brain. At least some of the detectors 30 are arranged to be spatially separated from the light source 20. One or more (e.g. each) of the light detectors 30 may be arranged to be sufficiently spaced apart from the light source 20 so that at least some of the photons of sample light from the light source 20 which is received at the light detector 30 will have penetrated into the subject's brain tissue. For example, the source-detector spacing may be selected so that the light detector 30 is arranged to receive sample light photons which have undergone multiple scattering events (e.g. which have scattered multiple times between source and detector as they travel through the subject's head 2). In other words, the source-detector spacings may be selected so that light detectors 30 are receiving deeply penetrating photons from the subject's brain tissue. Such photons may have longer time of flights from source to detector, as compared to photons which penetrate more shallowly and undergo fewer scattering events.

The detectors 30 may be arranged to be arranged to be spatially proximal to each other on the subject's scalp. The arrangement of the detectors 30 may be selected so that the detectors are imaging a similar region of the subject's brain. For example, the detectors 30 may be located within a threshold distance of each other on the subject's scalp so that they data they obtain may be averaged (e.g. to provide average values for the same volume of the subject's brain). That is, the detectors 30 may be arranged to spatially probe the same volume of tissue within the subject's brain. For example, the detectors may be arranged to be within one attenuation length for the tissue of each other (e.g. within the sum of the absorption and scattering coefficients).

The light source 20 is arranged to generate light and to direct this light towards the subject's scalp and the light detectors 30 (via the reference channel(s)). The light splitter 24 is arranged to receive light generated by the light source 20 and to split this light into two channels: (i) towards the subject's scalp using the sample delivery channel 25 and sample delivery probe 25a, and (ii) to the light detectors 30 using the reference delivery channel 26 and reference receiving channels 36. The splitter is configured so that the majority of the light is directed towards the subject's scalp. For example, the splitter may be a 90:10 splitter, or a 99:1 splitter. The sample delivery channel 25 is arranged to receive sample light from the splitter, and to deliver this sample light towards the subject's scalp (via the sample delivery probe 25a). The reference delivery channel 26 is arranged to receive reference light from the splitter, and to deliver this sample light to the detectors (via the reference receiving channels 36).

Each of the reference delivery connections 28 is arranged to deliver some of the reference light travelling along the reference delivery channel 26 to one of the reference receiving channels 36. Each of the reference receiving channels 36 is arranged to deliver the reference light to its light detector 30. The sample receiving probe 35a is arranged to receive sample light from the subject's brain tissue. The sample receiving probe 35a may focus the received sample light onto the sample receiving channel 35. The sample receiving channel 35 is arranged to deliver received sample light to its light detector 30. The sample receiving probes 35a may be arranged in close proximity to each other on the subject's scalp.

Each detector is arranged to receive two inputs: (i) reference light directly from the light source 20, and (ii) sample light indirectly from the light source 20 (e.g. which has travelled via the subject's brain tissue, as well as through their scalp skin and skull). For example, each detector may comprise two or more input ports. A first input port of the detector may be coupled to the reference delivery channel 26 for that detector. A second input port of the detector may be coupled to the sample delivery channel 25 for that detector. The detector is arranged to combine reference light with sample light (as an interferometer). The detector and controller 40 are arranged to determine one or more properties of the subject's brain tissue based on this combination of reference light and sample light (as will be described in more detail below).

The light source 20 is configured to provide wavelength swept emission of light. For this, the light source 20 may be configured to produce a series of emissions of pulses of light. During each pulse, the wavelength of light may be “swept” through a range of wavelengths. For example, the sweeping may be in the form of a chirped pulse. Light will be emitted at a plurality of different wavelengths during one pulse. For example, the wavelength may continually increase or decrease during one pulse (the rate of change of wavelength may be constant, or it may be variable). The series of chirped pulses may be contiguous (e.g. with a zero inter-pulse time interval). The light source 20 may be configured to successively emit a series of pulses, with each pulse having a wavelength sweep. However, it will be appreciated that the light source 20 need not provide continuous sweeping. For example, the light source could be tuned in steps rather than continuously, such that the light source 20 emits light at different wavelengths in different time intervals (e.g. discrete time intervals for emission at each of a plurality of wavelengths). The light source 20 may sweep unidirectionally (e.g. only increasing or decreasing in wavelength during one wavelength sweep), or it may sweep bidirectionally (e.g. both increasing and decreasing in wavelength during one wavelength sweep). Unidirectional sweeping can be beneficial as it increases the number of detected photons per sweep.

The controller 40 may be configured to selectively control the wavelength sweeping of the light source 20. The light source modifier 22 is arranged to control the wavelength emission of light from the light source 20. For instance, the light source modifier 22 may be arranged to apply a selected current (or voltage) to the light source 20 to select a wavelength emission from the light source 20. The wavelength sweeping of the light source 20 may be controlled by using the light source modifier 22 to apply a corresponding electrical signal to the light source 20. The controller 40 may be arranged to control application of a current/voltage to the light source 20 using the light source modifier 22 to provide a selected pattern for the wavelengths of light emitted by the light source 20.

The light source 20 may be controlled to wavelength sweep according to a selected pattern for the sweeping. For example, the light source 20 may sweep through a selected range of wavelengths of light and/or the light source 20 may sweep through wavelengths of light according to a selected sweep profile (e.g. linear increasing, sinusoid, triangular etc.). For example, the light source 20 may sweep according to a selected sweeping rate, or a selected total sweeping time. The light source 20 is configured to wavelength sweep light so that during one wavelength sweep, light will be directed towards the subject's brain tissue through the sample delivery channel (and to the detectors via the reference channels) at each of a plurality of different wavelengths. The wavelength of light emitted by the light source 20 will vary over time. As such, an indication of the time at which light was emitted from the light source 20 may be determined based on a wavelength of that light.

The light source 20 may be configured to sweep through a selected wavelength range. For example, the light source 20 may be configured to sweep in optical frequency over a range of 50 GHz. For example, this may enable the light source 20 to emit modulated light at a plurality of different wavelengths between e.g. 829.94 nm and 830.06 nm when centred on 830 nm for example or between 1309.857 nm and 1310.143 nm when centred on 1310 nm for example. The light source 20 may be configured to sweep through a wavelength range of at least 0.025 nm, such as at least 0.05 nm, such as at least 0.075 nm, such as at least 0.1 nm, such as at least 0.11 nm (e.g. about a wavelength on which it is centred). The light source 20 may have a high output power, a long coherence time, and broad mode-hop free wavelength tuning. The light source 20 may have a relatively narrow linewidth and a longer coherence length, e.g. because the light source 20 will not sweep over particularly large bandwidths.

Light sources of the present disclosure may be configured to provide emission of high coherence light, e.g. substantially coherent light. It will be appreciated that the light source may not both emit perfectly coherent light and also provide wavelength swept emission of light, e.g. because light at different wavelengths will change phase at different rates. Light sources of the present disclosure may be controlled to sweep through a wavelength range which is relatively narrow compared to their absolute wavelength. In other words, the difference between the maximum and minimum wavelengths for one wavelength sweep will be relatively small compared to those wavelengths. Each light source may be configured to emit light (i.e. an electric field) which does not have much change in its phase over time.

The iNIRS system of the present disclosure will receive sample light and reference light, both of which originated from the same light source. The light sources of the present disclosure are configured to provide wavelength swept emission of sufficiently coherent light, such that the sample light and reference light, as received at the optical detector, will be in relatively similar phase to each other. As such, the combination of sample and reference light will give rise to substantially constructive interference between the two waves (e.g. the two streams of light wave will have sufficiently similar phases so that the resulting combined light signal will contain a constructive combination of the two light waves). In other words, the coherence length of the light source may be such that the multiple scattering in the tissue will not reduce the coherence or fringe contrast below a noise floor for the measurement.

For example, each light source of the present disclosure may comprise a laser. The laser may be selected based on its coherence length, e.g. to enable the constructive interference described above between sample light and reference light to occur. In other words, the iNIRS system may be arranged so that a maximum expected time of flight delay for sample light photons (received at the optical detector which have travelled through the subject's brain tissue) relative to reference light photons (received at the optical detector which have travelled along the one or more reference channels) is within a coherence time period for the laser (e.g. the difference in optical path length between the sample and reference light is within the coherence length of the laser).

Within this coherence time period, the phase of light emitted by the laser is approximately stable (despite changes in the wavelength of light being emitted). As such, there may be no loss in amplitude for combined light signals at the optical detector (e.g. the interference occurring at the detector may be substantially completely constructive).

For example, the iNIRS system may be configured to have a coherence length or range of approximately 50 m in air—e.g. the light sources may be selected which have a coherence length of between 50 and 100 m (a coherence time period of between 166 ns and 333 ns). It will be appreciated that this particular range is not intended to be limiting, rather it is illustrative of the approximate range for the light source. The light source may be selected so that it has a coherence length which is two or more times greater than the maximum expected optical path length difference, e.g. the coherence length may be three or four or more times greater. Having a light source with a coherence length which is much greater than the optical path length may increase accuracy for measuring sample light photons which have undergone a large number of scattering interactions within the subject's brain tissue.

The iNIRS system 10 is arranged so that the source-detector path lengths for reference and sample light are different. In other words, the iNIRS system 10 is arranged so that an average, or expected, optical path length for light travelling from the light source 20 to each detector via the subject's brain tissue will be different to the optical path length for light travelling from the light source 20 to said detector via reference channel(s).

As will be appreciated in the context of the present disclosure, photons of sample light which are directed towards the subject's brain tissue may travel from the light source 20 to a light detector 30 via a practically infinite number of different paths. A photon of sample light may undergo a large number of scattering events, and so follow a very tortuous path, between the sample delivery probe 25a and the sample receiving probe 35a. The iNIRS system 10 is arranged to provide neuroimaging and analysis based at least in part on activity in the subject's brain tissue. The time of flight for a sample light photon from light source 20 to light detector 30 will of course increase as the path length it takes increases. As such, a photon which travels a longer path, and penetrates deeper into the subject's brain tissue, will take even longer to arrive at the light detector 30. The longer the time of flight for a sample light photon, the deeper that photon is likely to have penetrated into the subject's brain tissue.

The iNIRS system 10 is arranged so that the shortest time of flight for photons of light to travel from light source 20 to light detector 30 will be for photons of reference light travelling along the reference channel(s). The sample light photons will have longer times of flight than this reference light. The sample light photons which penetrate the deepest into the subject's brain tissue are likely to be those which have the longest time of flight to the light detector 30.

The iNIRS system 10 is arranged to determine a distribution of time of flight (‘DTOF’) for sample light photons. For this, a temporal point spread function (‘TPSF’) may be determined, which is similar to the DTOF, but which includes an Instrument Response Function (‘IRF’) which can subsequently be filtered out (e.g. deconvolved and/or subtracted) using post-processing. Each determined DTOF may provide a distribution showing the time of flight for all sample light photons which were incident on the light detector 30 at a given moment in time. The DTOF may contain an ensemble average representing a large number of incident photons (in each of a plurality of different TOF bins). The intensity for each TOF bin will provide an indication of the number of incident photons at that TOF. A phase of a TOF bin (e.g. obtained using a Fourier analysis) may represent an average phase for all of the photons arriving in that TOF bin. As described in more detail below, numerous properties of the subject's brain tissue may be determined based on DTOF data obtained for the subject's brain tissue. To obtain such data, the iNIRS system 10 is configured to obtain interferograms for the combination of reference light and sample light, as received at a given light source 20.

The iNIRS system 10 is arranged so that each of the light detectors 30 receives both sample light and reference light, and combines the two using a light combiner. For example, each of the detectors may provide an interferometer assembly (in combination with the sample and reference light channels) configured to combine the reference light and the sample light to obtain an interference pattern (an interferogram).

The light source 20 is configured to emit substantially coherent light. The resulting interference pattern for light from the light source 20 (as obtained at each detector) may comprise a combined signal having components at beat (or intermediate/difference) frequencies corresponding to the difference in wavelength between: (i) wavelengths of the photons of sample light received at the light detector 30 at a given instance in time, and (ii) the wavelength of photons of reference light received at the light detector 30 at that instance in time. The reference light at a single sampling interval should be substantially narrow and uniform wavelength as limited by either the intrinsic linewidth of the laser and the optical frequency sweeping rate, as the received photons of reference light will have travelled the same distance (through reference channels) to the light detector 30 from the light source 20. The sample light will include photons at different wavelengths, where each wavelength of sample light will correspond to the time of flight for that photon and its unique path through the tissue (due to the wavelength sweeping of the light source 20). The resulting interferogram will therefore contain a plurality of different beat frequencies (due to the different differences in wavelength). The higher beat frequencies may correspond to photons with higher times of flight (deeper penetrating photons) in the event that the sample path is longer than the reference path.

Each light detector 30 comprises a light combiner arranged to combine the light, and to provide said combined light (which may include one or more components corresponding to beat frequencies) to signal processing circuitry. Each of the detectors may comprise a square law detector. For example, each detector may be configured to create an interference pattern based on the difference in optical frequencies of the incident sample and reference electric fields, e.g. wherein the intensity or power detected is proportional to the square of the incident electric field and the incident electric field is the sum of the sample and reference electric fields. Such that the intensity or power detected (in the form of a photocurrent) is equal to the square of the sum of the incident sample and reference electric fields. Such detectors may comprise photodiodes, avalanche photodiodes and/or fast linescan cameras, streak cameras and fast CCD or CMOS sensors. The detector may be a high bandwidth detector. For example, the detector may be configured to resolve interference fringes at 100 Mhz or more, such as up to 1 GHz. The detector may comprise a single speckle detector. For example, optical fibres used in the detector may be single mode. If a multi-mode detector is used, then the detector may comprise an array of square-law detectors, such as a photodiode array, focal plane array, fast linescan camera or fast CCD array. The detector may also comprise a balanced detector array. For example, the balanced detector array may be configured so that the reference light and the scattered light are combined and split (e.g. evenly) onto a pair of out-of-phase detectors such as with a 4-port (2-in, 2-out 50:50 ratio) fibre coupler or a beamsplitter cube. A balanced detector may enhance signal to noise of the detected signal by rejecting incoherent portions of the signal. The balanced photodetector may also fully utilise all light transmitted through the interferometer and suppress common noise, such as laser intensity noise.

In other words, each light detector 30 may comprise a light combiner configured to combine the sample and reference light to provide a combined light signal. The iNIRS system 10 is arranged to process that combined light signal to obtain an indication of the intensity of light incident on the light detector 30 at a given moment in time (e.g. through use of optical heterodyning and/or balanced detection). The iNIRS system 10 is arranged to obtain a plurality of such indications, e.g. the light detector 30 may be arranged to repeatedly obtain indications of the intensity of light incident on the light detector 30. In other words, the iNIRS system 10 is arranged to measure a phase or frequency shift between photons of light in the two inputs to the detector (reference and sample), and to attribute such differences to properties of the intervening brain tissue for the sample light.

Each obtained interferogram may be Fourier analysed (e.g. using an FFT or IFT) to obtain an indication of a DTOF for the sample light photons incident on the light detector 30. When a square-law detector is used, the intensity at the detector may be proportional to the square of the summed electric field intensity. The rate of change of the optical frequency multiplied by time delay may give rise to a frequency of the interference fringes present in each interferogram. In other words, a Fourier Transformed interferogram containing a plurality of beat frequencies may be used to indicate the photon time of flights associated with those beat frequencies present in the interferogram.

To obtain this data of DTOF for sample light photons, the light detector 30 is arranged to combine the two light inputs (sample and reference) into a combined light beam. The light detector 30 is arranged to convert the combined light beam into an electrical signal representative of the combined light signal. The iNIRS system 10 includes at least one digitiser arranged to receive electrical signals indicative of combined light signals and to convert those electrical signals into digital data representing said combined light signals. This digital data may be processed to obtain one or more different properties of the subject's brain tissue for neuroimaging and analysis.

One example of an arrangement for converting received light signals into digital data is shown in Inset A of FIG. 1. Inset A shows an arrangement of components that may be used as a light detector 30 of the present disclosure. As also shown in the iNIRS system 10 of FIG. 1, the detector 30 receives two inputs: (i) reference light which has travelled along reference delivery channel 26 and reference receiving channel 36, and (ii) sample light which has been received through the sample receiving probe 35a and delivered to the detector via the sample receiving channel 35.

As shown, the detector may include a light combiner and splitter 301, a first light channel 302a and a second light channel 302b, a balanced photodetector 303, a transimpedance amplifier 304, an amplifier 305, an analogue to digital converter (‘ADC’) 306. The ADC 306 is arranged to provide a digital signal output 307.

The light combiner and splitter 301 is coupled to both the reference receiving channel 36 and the sample receiving channel 35. The light combiner and splitter 301 is arranged to receive both the sample and reference light, and to combine the two to provide a combined light signal. The light combiner and splitter 301 is arranged to split that combined light signal onto two separate channels: the first light channel 302a and the second light channel 302b. For example, this may be a 50:50 split (or there or thereabouts). The first light channel 302a and second light channel 302b are coupled to a balanced photodetector 303. Each light channel directs light towards an associated photodiode. The balanced detector is arranged to provide an output based on a difference between outputs from the two photodetectors. The two photodetectors will typically be provided so that the beat signals on each photodiode are 180° out of phase with each other, and so the coherent AC terms will combine positively with each other. The balanced photodetector 303 is arranged to output a current corresponding to the difference between the two photodetector output currents. The balanced photodetector 303 may remove any unwanted DC terms from this signal, such as slow fluctuations emanating from the light source 20 or other common-mode effects such as noise.

The light detector 30 is configured to use a current to voltage converter to convert the current output from the balanced photodetector 303 into a corresponding voltage. As shown in Inset A of FIG. 1, the converter may comprise a transimpedance amplifier 304. The voltage output from the transimpedance amplifier 304 is then amplified using the amplifier 305. The amplifier may be used to scale the output signal to the full range of the ADC and limit the electronic frequency of the circuit to further maximise the SNR. This amplified voltage is then provided to the ADC 306 to be digitised. The ADC 306 comprises a digitiser having sufficient bandwidth so that the full signal bandwidth containing time of flight information may be digitised without attenuation. For example, the digitiser bandwidth may be at least as large as the bandwidth for the combined light signal. For example, the digitiser may be selected to have a sampling rate high enough so that the Nyquist criterion is met for the bandwidth of the signal to be processed. The digitiser may be provided as part of each light detector 30, or the digitiser may be part of the controller 40, and the controller 40 may be coupled to each of the light detectors 30 to receive electrical signals therefrom which are to be digitised. For each combined light signal, a digital signal output 307 will be provided which gives a digital representation of that combined light signal (and thus of the sample light incident on the light detector 30 at the moment in time when that combined light signal was generated and measured).

The iNIRS system 10 is configured to obtain a plurality of digital signal outputs 307 indicative of sample light incident on light detectors 30. In particular, each light detector 30 is configured to repeatedly combine light signals (sample and reference) for providing digital signal outputs 307 representative of each combined light signal. For example, for each light detector 30, a time series of digital signal outputs 307 may be obtained, wherein each subsequent digital signal output 307 is for a subsequent point in time at which a combined light signal was obtained and measured (and the digital signal output 307 represents that combined light signal as obtained and measured). As described above, each of these signals may be indicative of a sample light DTOF for that point in time at that detector.

In other words, the iNIRS system 10 is configured to obtain a plurality of time-ordered DTOFs for each of a plurality of different light detectors 30. The digitiser may provide a digital output indicative of the different measurements, and this digital output may optionally be processed in a number of ways to provide the DTOF data. Examples of such steps will now be described.

The controller 40 may be configured to receive raw digital interferogram data (e.g. data representative of the interferogram obtained by converting the combined light signal into digital data). This raw interferogram may be divided into individual sweeps for the wavelength swept emission from the light source 20. For example, the sweep rate of the light source 20 and a time at which the first sweep commenced may be used to determine the sweep cycles. The data may then be divided into groups, where each group represents an individual sweep. An optional Hilbert transform may be performed on the data at this stage. Data windowing may be performed (e.g. with a Hann or Blackman-Harris window) to reduce sidelobes in the data. A Fourier analysis may be performed on the data, either inverse or normal. For instance, an inverse Fourier Transform may be performed. The Fourier analysis may be performed for each wavelength sweep of the light source 20. The resulting data may be in the form of a series of Temporal Point Spread Functions (‘TPSF’), with each TPSF corresponding to an associated wavelength sweep. The TPSF data may be processed to remove an Instrument Response Function (‘IRF’) therefrom to provide the DTOF data.

In other words, the iNIRS system 10 may be configured to determine a time-ordered series of time of flight distributions for sample light photons incident on each of a plurality of different detectors. This DTOF data may be processed to provide information relating to a number of different physical properties of the subject's brain tissue. The DTOF data may be used to determine optical properties of the medium through which the sample light has travelled. Each TOF bin in a DTOF may represent a selected volume within the subject's brain, and each DTOF represents a total volume of tissue probed by the photons (e.g. each DTOF may represent a weighted average of properties of the brain tissue, as well as other tissues through which the photons have travelled such as scalp, skull etc.). The optical properties include scattering and absorption properties for the subject's brain tissue. The DTOF data may be used to determine dynamic properties of the subject's brain tissue, such as how particular properties vary over time. This includes how the optical properties evolve over time, as well as properties indicative of movement within the subject's brain tissue (e.g. due to the flow of blood). The iNIRS system 10 may be configured to Fourier transform (e.g. FFT) the obtained TOF distributions to obtain gamma data, where the dynamical signals may be obtained from temporal changes in the gamma signals.

The time-ordered series of DTOFs may be considered to correspond to a surface of data in a 3D volume. That surface may represent the DTOF for each subsequent DTOF (ordered in time), and so the surface shows each individual DTOF, as well as the evolution of the DTOFs over time. This surface may provide a wealth of data from which properties of the subject's brain tissue may be determined. An analysis of the temporal fluctuations in (e.g. decay of) DTOF values over time may provide an indication of one or more dynamic properties of the subject's brain tissue. That is, the decay analysis may be used to identify that one or more properties within the subject's brain tissue are changing, as well as optionally identifying the rate at which these properties are changing (e.g. using the decay rate). The order of the decay of DTOF (e.g. decaying with t, t² etc.) may be used to determine one or more properties of the type of motion (e.g. diffusion or flow).

The controller 40 may store data which correlates time of flight for a sample light photon to an indication of average path trajectory for that photon. This may include an indication of the depth of the penetration into the subject's brain tissue for that photon, and/or an indication of the region(s) of the subject's brain tissue through which that photon travelled from light source 20 to light detector 30. The controller 40 may be configured to process the DTOF data by dividing this data up into selected time of flight bins. Within each TOF bin, the data may provide depth-resolved evolution data for the subject's brain tissue. That is, as the TOF may be associated with certain penetration depths or regions, each TOF bin may contain data showing properties associated with a certain penetration depth or region. The evolution of data within each TOF bin may therefore provide an indication of how one or more properties of the subject's brain tissue are evolving. For example, where the evolution suggests a change in movement (e.g. a flow of blood), that movement may be identified, as may the region in which that movement is occurring. For this, a TOF-resolved decay slope may be used to identify how the curve is decaying over time for specific TOFs (e.g. for specific penetration depths/regions).

In other words, the iNIRS system 10 is configured to perform an autocorrelation in which DTOFs for successive wavelength sweeps are combined to assess fluctuations in the light field at the light detector 30 over time. The fluctuations may be quantified due to relevant fluctuations in DTOFs over time. The fluctuations may also be depth-resolved, by identifying the relevant TOFs at which those fluctuations are occurring (and thus the relevant penetration depths/regions).

The controller 40 may be configured to process the data received from the light detector(s) 30 to provide time of flight information with depth-resolved autocorrelations for the subject's brain tissue. The controller 40 may be configured to use this information to obtain an indication of a plurality of different properties of the subject's brain tissue, such as intracranial pressure (‘ICP’), blood flow index, artery elasticity, etc. These properties of the subject's brain tissue may be used for a plurality of different forms of neuroimaging and analysis, such as in a brain-computer interface, for imaging regions of the brain to identify potential localised injury or strokes, and/or to monitor neuro responses to substances, such as drugs.

### Intracranial Pressure (‘ICP’)

As described above, the iNIRS system 10 includes an optical interferometer arranged to combine sample light with reference light to provide combined light signals. The system 10 may detect frequencies of light received by the detector. The received light (i.e. the combined light signals) will include light at a plurality of beat frequencies between sample light and reference light. The iNIRS system 10 also includes signal processing and conversion circuitry coupled to the detector 30 and configured to produce combined light signal data from the combined light signals (i.e. to produce data indicative of the received combined light signals).

The controller 40 of the iNIRS system 10 is configured to process the combined light signal data to obtain time of flight data comprising a plurality of time of flight distributions for photons of sample light reaching the optical detector 30 from the light source 20. The controller 40 may be configured to process the combined light signal data to obtain a data surface containing a plurality of temporally sequential time of flight distributions (e.g. a time-ordered series of DTOFs). The controller 40 may be configured to process this data surface to obtain an indication of an autocorrelation decay. Each autocorrelation may provide an indication of how similar a first DTOF distribution is to a DTOF distribution which preceded it (or even how similar a portion of the first DTOF distribution is to a corresponding portion of the subsequent DTOF distribution, e.g. with the portion representing a certain time-of-flight, ‘TOF’, bin). The autocorrelation decay may provide an indication of how the autocorrelations vary over time, e.g. of how the autocorrelations are changing. In other words, the controller 40 may be configured to obtain an indication of any temporal fluctuations in the DTOF distributions obtained for the subject over time. The DTOF distributions may contain data for each of a plurality of TOF bins. Some of these TOF bins (typically those with longer times) may represent volumes of tissue within the subject's brain. The controller 40 may be configured to identify temporal fluctuations associated with one or more volumes of tissue within the subject's brain.

The controller 40 is also configured to obtain an indication of scattering and absorption coefficients for the subject's brain tissue. The controller 40 may determine the coefficients using time of flight data (e.g. based on one or more of the obtained DTOFs). For example, the controller 40 may determine the coefficients based on one or more statistical moments (e.g. mean/variance) for a DTOF (or TPSF) distribution (or a plurality of such distributions), and/or based on a shape of the DTOF (or TPSF) distribution (or a plurality of such distributions).

The controller 40 is configured to determine a cerebral blood flow index for the subject based on the obtained scattering and absorption coefficients and the autocorrelation decay. For this, the controller 40 may be configured to attribute temporal fluctuations in a volume of the subject's brain to movement of blood in that region. The cerebral blood flow index may provide a relative measure of movement occurring within tissue in the subject's brain.

The controller 40 is configured to obtain cerebral blood flow data for the subject's brain tissue (i.e. for tissue within the subject's brain). The cerebral blood flow data may be determined based on detected changes occurring within the subject's brain tissue. The controller 40 may be configured to detect changes relating to blood within the subject's brain tissue. These changes may relate to the flow of blood through the subject's brain tissue. In other words, the controller 40 may be configured to detect an indication that blood is moving in a given region of the subject's brain that region by determining that decorrelation is occurring for subsequent DTOF distributions.

As set out above, the controller 40 may be configured to determine a Cerebral Blood Flow index (‘CBFi’) for the subject's brain tissue. The CBFi may provide an indication of movement occurring within the subject's brain. For example, the CBFi may provide a relative measure that describes movement occurring within the subject's brain. The movement may be attributed to blood moving (e.g. flowing) through the subject's brain. The CBFi provides a relative measure. The CBFi may contain an indication of mean velocities in bulk tissue. For example, the CBFi for a given volume within the subject's brain may provide an indication of a mean velocity of movement occurring within that volume. That movement may be attributed to movement of blood, and so the CBFi may provide an indication of mean blood flow velocity. For example, an increased value for CBFi may indicate an increased velocity occurring within the volume of the brain.

The iNIRS system may be sensitive to all forms of movement of blood through the subject's brain. For example, the blood flow being monitored by the iNIRS system 10 may be for vessels within the brain tissue, such as for blood perfused in microvasculature of the subject's brain tissue, and/or it may be for Brownian motion type movement through the subject's brain tissue (rather than the flow of blood through larger tube-like structures, such as veins or arteries). It will be appreciated that the precise form of blood movement detected by the iNIRS system will depend on the volume of tissue being examined by the iNIRS system.

The iNIRS system 10 may be configured to measure an indication of perfusion pressure for blood flow within the brain tissue. The CBFi may provide an indication of the behaviour of blood as it flows through such regions of the subject's brain tissue.

The iNIRS system 10 may be configured to obtain data for the subject's brain tissue at a rate sufficiently high enough to identify changes in the flow of blood through the subject's brain tissue. In particular, the iNIRS system 10 may be configured to obtain data at a rate high enough to identify changes in the subject's blood flow which are brought about due to the beating of their heart (e.g. to identify systolic and diastolic properties for the blood flow). For example, the iNIRS system 10 may be configured to obtain data at a rate of at least 10 Hz, such as more than 20 Hz, e.g. between 10 and 100 Hz.

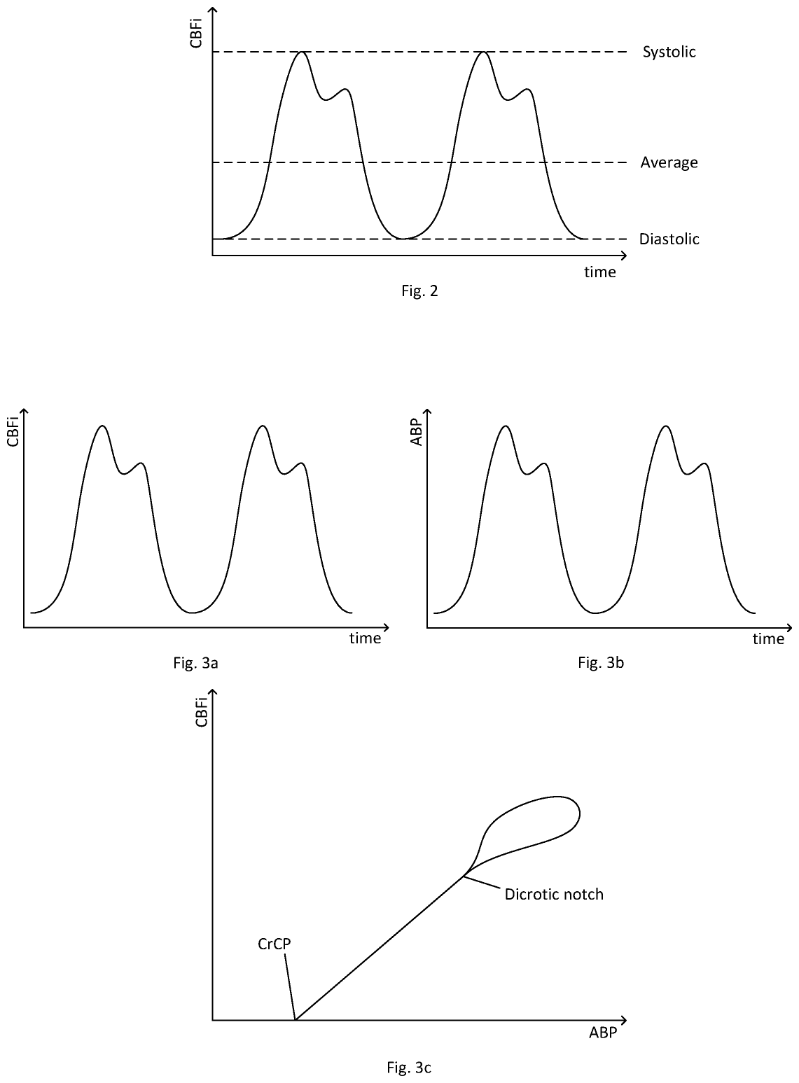

The controller 40 is configured to obtain cerebral blood flow data (i.e. data containing an indication of blood flow within the subject's brain tissue) which contains an indication of one or more pulses of blood flow through the subject's brain tissue. The controller 40 may determine the CBFi for the subject's brain tissue, wherein the pulses of blood flow through their brain tissue are evident in the determined CBFi for their brain tissue. An example of such a determined CBFi is shown in FIG. 2.

FIG. 2 shows an example of a determined CBFi for a subject's brain tissue evolving over time. In FIG. 2, there are two pulses shown. The pulses occur in a cyclical and repetitive manner (although each individual pulse may vary in size and shape, and the frequency at which pulses occur may also change). Typically, each pulse will have a diastolic CBFi value (e.g. lower CBFi value) and a systolic value (e.g. higher CBFi value). During one pulse, the CBFi may start at its diastolic value (its lowest value), and then rapidly increase up to its systolic value (its highest value). The CBFi will then drop back towards its diastolic value from the systolic value, but the CBFi may temporarily increase (or at least begin to decrease at a slower rate) at least once as it drops from systolic to diastolic values. Once back at the diastolic value, the process will repeat again (due to the beating of the subject's heart).

The iNIRS system 10 is configured to use optical interferometry to obtain measurement signals from which the controller 40 of the iNIRS system 10 may obtain cerebral blood flow data indicating this behaviour for the blood flowing through the subject's brain tissue (e.g. the controller 40 may obtain the temporal evolution of the CBFi values as shown in FIG. 2).

As will be described in more detail below, the controller 40 is configured to process the cerebral blood flow data to determine an indication of ICP for the subject's brain tissue. The controller 40 may be configured to determine ICP based on one or more properties of the pulsatile waveform for the blood flow. For this, the controller 40 may use any of a number of suitable properties. In examples, the properties may describe the shape of the pulsatile waveform, or other relative measures relating to the pulsatile waveform.

The volume in which the subject's brain tissue is provided will be constrained by their skull. The skull will typically be very rigid, and so the subject's brain tissue is provided within a fixed internal volume of the skull. This internal volume will not change, and so, as the pressure within the skull increases, the matter in that internal volume will become more compressed (and vice-versa). This will also apply to pulses of blood flowing through the subject's brain tissue. For blood to flow through the subject's brain tissue, some of the brain tissue will have to be compressed to make way for the blood to get through. The amount by which their brain tissue may be compressed to accommodate blood flow will depend on their ICP. For a given pulse of blood flow through the subject's brain tissue, the amount by which the brain tissue compresses to accommodate a pulse of blood flow will decrease with increased ICP (as with increased ICP the brain tissue will already be more compressed). In turn, some of the blood flow may be slower (e.g. due to increased resistance to its motion), and/or the quantity of blood flowing per unit time may be reduced.

The controller 40 is configured to determine an indication of the influence of ICP on the pulsatile waveform for blood flow through the subject's brain tissue using the cerebral blood flow data. The controller 40 may be configured to identify changes in the pulsatile waveform, e.g. its shape changing and/or its maximum/minimum values changing, and to use this information to determine an indication of the subject's ICP. For example, the controller 40 may be configured to determine an indication of ICP based on maximum and/or minimum CBFi values for the pulses of blood flow through the subject's brain tissue, and/or based on any differences between these values.

With reference to FIG. 2, an example method of determining ICP will now be described. As set out above, FIG. 2 shows the CBFi values for the pulsatile waveform of pulses of blood flow through the subject's brain tissue, as determined using the iNIRS system 10 of the present disclosure. The controller 40 is configured to determine, for each pulse, an indication of its systolic (e.g. highest) CBFi value and its diastolic (e.g. lowest) CBFi value. Both of these values are illustrated in FIG. 2. Additionally, the controller 40 is configured to determine an average CBFi value for each pulse (also shown in FIG. 2). The controller 40 is configured to determine a Cerebral Blood Flow Pulsatility Index value based on the diastolic, systolic and average CBFi values for the pulse. For example, the CBFi Pulsatility Index may be determined as the difference between the systolic and CBFi diastolic values divided by the average CBFi value for each pulse. The controller 40 may be configured to determine an indication of ICP based on the determined CBFi Pulsatility Index. For example, the controller 40 may utilise stored data (such as in a look-up table) which links the CBFi Pulsatility Index value to an ICP value, e.g. this may use a known regression relationship between CBFi Pulsatility Index and ICP.

Another example method of determining ICP will now be described with reference to FIGS. 3a to 3c. For this method, an additional data stream is used in combination with the CBFi data obtained by the iNIRS system 10. The additional data stream contains data indicative of one or more pulses of blood flow for a different region of the subject's body to their brain tissue, hereinafter referred to as extracerebral blood flow data. The extracerebral blood flow data will be from a region of the subject's body which is not subjected to the same fixed volume constraints that are imposed by the subject's skull. The method involves comparing one or more properties of pulse waveforms for cerebral blood flow with corresponding properties for the pulse waveforms for extracerebral blood flow. An indication of ICP for the subject's brain tissue may then be determined based on differences between their cerebral and extracerebral pulses of blood flow (e.g. from differences in the shape and/or values for the pulse waveforms).

FIG. 3a is similar to FIG. 2 in that it shows the CBFi values for the pulsatile waveform of pulses of blood flow through the subject's brain tissue, as determined using the iNIRS system 10 of the present disclosure. FIG. 3b shows a similar looking graph to FIG. 3a, however, this graph shows pulses of extracerebral blood flow. The extracerebral blood flow is for a portion of the subject's body not subject to a fixed volume constraint (as is the case within the subject's skull). In such an extracerebral region, each pulse of blood flow will have a pulsatility waveform which shows less influence from pressure exerted onto that blood vessel from surrounding tissue. In other words, for a blood vessel in the extracerebral region, the tissue which surrounds that vessel may only be subject to much smaller fluctuations in local pressure as compared to those which may occur inside of the subject's skull (i.e. where an increase in pressure cannot be accommodated by a corresponding increase in volume). For example, the extracerebral blood flow may be for the flow of blood through a subject's veins or arteries, such as those in their scalp.

In the example of FIG. 3b, the graph shows a plot of arterial blood pressure (‘ABP’) over time (rather than for cerebral blood flow index as in FIG. 3a). The ABP pulse waveforms (FIG. 3b) may appear relatively similar to those for CBFi (FIG. 3a). The controller 40 may be configured to temporally align corresponding pulse waveforms for the cerebral and extracerebral pulses of blood. For example, it will be appreciated that the pulse timings may be offset for different regions of the body, and so the controller 40 may be configured to align pulses from one region with corresponding pulses in the other region (so that a comparison of the two waveforms is associated with the same pulses, even if those pulses were measured at slightly different periods in time).

In one example, the controller 40 may be configured to perform a comparison between different values for the two pulses (e.g. diastolic, systolic and/or average values), such as in the manner outlined above. In which case, the controller 40 may use the values for the extracerebral blood flow to provide an indication of a baseline pulsatility index for pulses of the subject's blood flow, with any differences between this baseline pulsatility index and the CBFi pulsatility index being attributed to the influence of ICP on the pulses of blood flow through the subject's brain tissue (e.g. with a similar regression analysis for converting differences in pulsatility index between cerebral and extracerebral blood flow into an indication of ICP).

Another example for determining ICP based on cerebral and extracerebral blood flow data is shown in FIG. 3c. In FIG. 3c, the cerebral blood flow data is plotted against the extracerebral blood flow data, which is shown as CBFi on the y-axis and ABP on the x-axis. The two data plots (i.e. data for the cerebral and extracerebral blood flow) are aligned on the graph, e.g. so that their linear regions substantially overlie each other (or at least are close to one another in the event that the data plots do not perfectly overlie each other). As can be seen in FIG. 3c, there is a linear region from where the line crosses the x-axis (labelled ‘CrCP’) up to a region (labelled ‘dicrotic notch’) where the data diverges and there is a looped region. As can be seen in FIG. 3c, the curve crosses the x-axis (i.e. CBFi value of 0) with a positive ABP value. The controller 40 of the iNIRS system 10 may be configured to process the cerebral and extracerebral data to identify the value of this point (the CrCP).

The CrCP represents a critical closing pressure for blood vessels in the subject's brain tissue. As set out above, the CBFi provides information relating to movement of blood flow through vessels in the subject's brain tissue. As the CBFi value decreases, this indicates that there is less movement of blood within the subject's brain tissue (e.g. a zero measurement for CBFi may represent an absence of any blood flow). The intersection on the x-axis in the graph of FIG. 3c represents the value for arterial blood pressure at which there is a zero CBFi (i.e. at which no blood is flowing through the vessel in the subject's brain tissue). This is referred to as a critical closing pressure, as it is a pressure at which small blood vessels in the brain ‘close’. In other words, the value for CrCP represents the conditions under which the pressure in that blood vessel is sufficiently low that the blood vessel is forced to close (due to external pressure being applied thereto).

The controller 40 is configured to determine what the value for the CrCP is for one or more of the blood vessels in the subject's brain tissue. The controller 40 is configured to determine an indication of ICP based on this value for CrCP. The comparison between the arterial blood pressure and the CBFi may provide an indication of blood pressure in the subject's artery (at the same/corresponding point during the pulsatile waveform) when the blood vessel in their brain tissue closed. An indication of ICP may be determined based on this information (i.e. based on the pressure at which the blood vessel ‘closed’). For example, the ICP may be determined as the external pressure required to be applied to the blood vessel to cause that blood vessel to close. This may include a vascular compliance term which represents the force applied from the vascular wall against the blood (e.g. to account for there being some blood pressure existing in the vessel, e.g. as per the subject's arterial blood pressure).

While this method is depicted in FIG. 3c, it will be appreciated that the controller 40 need not produce corresponding graphs (these are shown to illustrate the method). The controller 40 may be configured to compare the extracerebral blood flow data (e.g. blood pressure data for one or more of the subject's veins/arteries outside of their brain tissue) and the cerebral blood flow data (e.g. CBFi values for one or more blood vessels in the subject's brain tissue). Based on this comparison, the controller 40 may be configured to identify a corresponding pressure (e.g. in the subject's vein/artery) at which the blood vessel in their brain tissue closes. The controller 40 may be configured to determine an indication of ICP based on this CrCP value (e.g. by determining a required pressure in the subject's brain which would cause the cerebral vessels to close).

In the methods where extracerebral blood flow data is obtained, it will be appreciated that any suitable device may be used to obtain this data. For example, the iNIRS system 10 may be provided in combination with a separate sensing element which is configured to obtain such data. The separate sensing element may be configured to measure blood pressure for a separate region of the subject's body—such as on one of their limbs—e.g. at an arm or on their hand/fingers. The sensing element may be configured to obtain data containing an indication of one or more properties of the pulsatile waveform in that region of the subject's body (e.g. including diastolic and/or systolic values, and/or average values). For example, the sensing element may be configured to obtain an indication of how the blood pressure changes over time during the pulsatile waveform for blood through the subject's vein/artery.

Additionally or alternatively, the iNIRS system 10 of the present disclosure may be configured to obtain both the cerebral blood flow data and the extracerebral blood flow data. For example, the iNIRS system 10 may contain a single source-detector channel which may be arranged to generate combined light signals from which data may be obtained which represents the cerebral blood flow and the extracerebral blood flow (e.g. with both pieces of data originally contained within one spectrogram/interferogram).

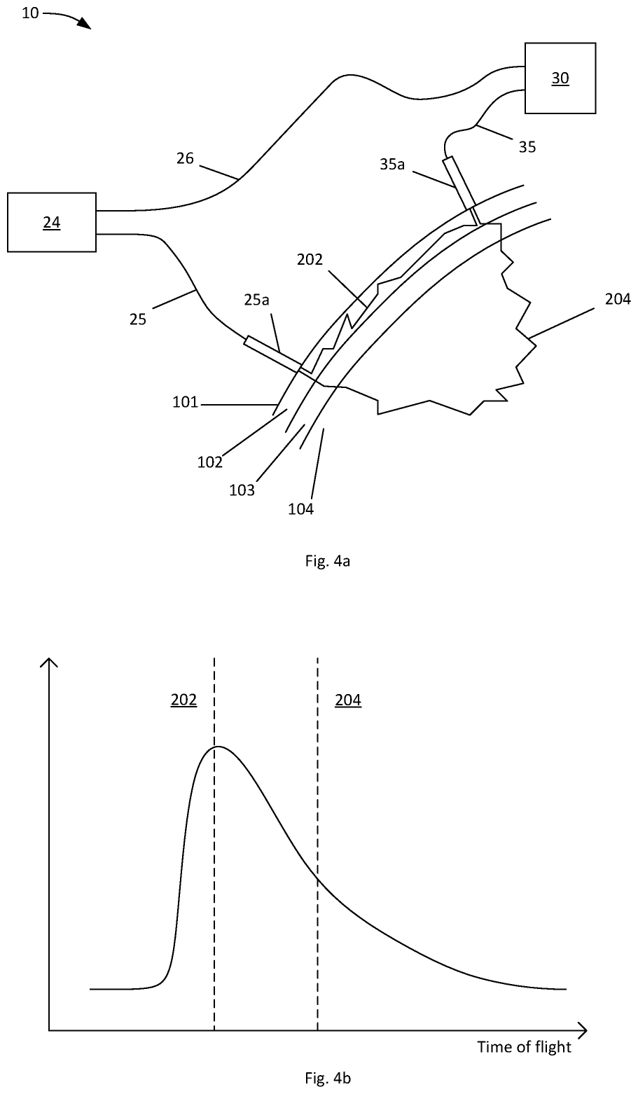

FIG. 4a shows an example iNIRS system 10 arranged to provide such functionality.

The iNIRS system 10 of FIG. 4a is similar to that of FIG. 1 described above, and so the repeat components shall not be described again. As set out above, the iNIRS system 10 is arranged for light from a light source 20 (not shown) to be split (by light splitter 24) onto a sample delivery channel 25 and a reference delivery channel 26. The optical detector 30 then receives reference light (from reference delivery channel 26) and sample light (from sample receiving channel 35). A sample delivery probe 25a and a reference delivery probe 35a are also shown.

FIG. 4a also shows different layers of the subject's head. The top layer is scalp skin surface 101. The second layer, which is beneath the scalp skin surface, is the scalp tissue 102. The scalp tissue will include a plurality of arteries and veins. Beneath the scalp tissue is the subject's skull 103, and located within their skull is brain tissue 104. The veins and arteries within the subject's scalp tissue are relatively unconstrained by their skull. That is, in this region of the subject's body, the veins/arteries are not impeded from expanding in volume by their skull (whereas blood vessels within the subject's brain tissue are). Therefore, an increase in blood pressure in the veins/arteries may provide a corresponding increase in volume in these regions.

For photons of light to reach the subject's brain tissue from the light source 20 (and through sample delivery channel 25 and sample delivery probe 25a), they must penetrate through the subject's scalp skin surface, the scalp tissue and the skull. For photons of light to reach the veins/arteries in the subject's scalp tissue, they must only penetrate through the subject's scalp skin surface (and relevant parts of the scalp tissue). Two example photon paths are shown for sample light reaching the detector 30 from the light source 20: shallow photon path 202 and deep photon path 204. The deep photon path between light source 20 and light detector 30 is much longer than the shallow photon path (as it travels deeper into the subject's brain tissue). For both photon paths, there are a number of scattering events which occur within the subject's head. In this example, a photon travelling along the shallow photon path will interact with at least one blood carrying region of the subject's scalp tissue (e.g. a vein or artery), and a photon travelling along the deep photon path will interact with at least one blood vessel within the subject's brain tissue.

The light source 20 will emit a much larger number of photons, and the paths they take through the subject's head from source 20 to detector 30 will vary. The sample delivery probe 25a and the sample receiving probe 35a are spatially arranged on the subject's scalp so that at least some of the sample light photons arriving at the detector 30 from the light source 20 will have travelled along shallow photon paths, and some will have travelled along deep photon paths. For example, the probes will be sufficiently spatially separated so that some deep photon paths occur.

FIG. 4b shows a time of flight distribution for the sample light photons received at the detector 30. As shown, the peak number of sample light photons occurs for a relatively short time of flight, and then for longer times of flight, the number of photons arriving decreases. As described above, the time of flight for sample light photons approximately corresponds to their penetration depth. That is, deeper penetrating photons will have longer times of flight than shallower penetrating photons.

The time of flight distribution shown in FIG. 4b contains sample light photons which have followed a shallow path (and may provide information relating to extracerebral blood flow) and sample light photons that have followed a deep path (and may provide information relating to cerebral blood flow). As shown in FIG. 4b, the shallow photon path photons have shorter times of flight than the deeper photon path photons. Within this single interferogram, the controller 40 may be configured to obtain information for both cerebral and extracerebral blood flow.

For this, the controller 40 is configured to demix the cerebral blood flow from the extracerebral blood flow. This may comprise the controller 40 separating cerebral blood flow data from extracerebral blood flow data based on time of flight data. For example, the controller 40 may be configured to attribute received sample light photons as relating to the subject's scalp tissue when those received photons have a time of flight below a first threshold. The controller 40 may be configured to attribute received sample light photons as relating to the subject's brain tissue when those received photons have a time of flight below a second threshold. For example, the first and second threshold may be different (e.g. so that photons which are likely to have scattered from the subject's skull have times of flight between the first and second thresholds). In other words, the controller 40 may be configured to obtain depth-resolved information for the received photons of sample light. This depth-resolved information may contain an indication of whether photons penetrated beyond the subject's skull or not. The controller 40 may select the non-skull penetrating photons for obtaining extracerebral data therefrom, and the skull-penetrating photons for obtaining the cerebral data therefrom.

The controller 40 may be configured to process the cerebral and extracerebral blood flow data separately. For example, the controller 40 may be configured to obtain time of flight data (e.g. which contains measured time of flight distributions), and to separate this time of flight data into different data streams: (i) an extracerebral data stream (for the shorter time of flight photons likely to be associated with the scalp tissue), and (ii) a cerebral data stream (for the longer time of flight photons likely to be associated with the brain tissue). Each data stream may be processed independently in the manner set out above-to obtain blood flow index data containing an indication of one or more pulses of blood through the relevant region of the subject's body (e.g. through veins/arteries in their scalp or blood vessels in their brain tissue).

The controller 40 may be configured to process the extracerebral blood flow data to obtain an indication of one or more properties of the pulsatile waveform for the extracerebral blood flow (e.g. to obtain blood flow index data for pulses of blood flow through veins/arteries in the subject's scalp tissue). The controller 40 may be configured to determine pressure data for the flow of pulses of blood through the subject's vein/artery (as evident from the blood flow index data obtained using the iNIRS system 10). For example, the controller 40 may store known conversion data for converting blood flow index values into corresponding pressure values (e.g. using a similar approach to the CBFi pulsatility index method described above). The controller 40 may be configured to obtain extracerebral blood flow pressure data, e.g. data containing pressure values for the pulses of blood flow throughout different time periods during each pulsatile waveform for the pulses of blood flow through the subject's scalp tissue. The controller 40 may use the extracerebral pressure data and the cerebral blood flow index data to determine an indication of the intracranial pressure (e.g. in the manner set out above in relation to FIGS. 3a to 3c, such as by identifying a CrCP value for the subject's brain tissue).

The iNIRS system 10 may thus be configured to obtain, through iNIRS measurements, both the cerebral and extracerebral data. The cerebral data may contain cerebral blood flow index data for one or more pulses of blood flow through blood vessels in the subject's brain tissue. The extracerebral data may contain blood pressure data for one or more pulses of blood flow through the subject's scalp vascular system. The controller 40 may be configured to process these two data streams obtained using the iNIRS system 10, and to determine an ICP for the subject based on these two data streams. For example, the controller 40 may be configured to align the obtained scalp tissue blood pressure data with the obtained cerebral blood flow index data, and to determine therefrom an indication of the extracerebral blood pressure value at which one or more blood vessels in the subject's brain tissue close. Based on this critical closing pressure, the controller 40 may determine the indication of ICP.

In the examples described above in relation to FIGS. 2 to 4, the iNIRS system 10 uses one light source and one detector (i.e. there is only one source-detector channel present). However, this should not be considered limiting. The iNIRS system 10 may include a plurality of light sources and/or a plurality of light detectors. Each light source may be coupled to a plurality of other detectors (e.g. via a plurality of reference channels). The plurality of light detectors may be collocated (e.g. they may be provided in the same region on the subject's scalp/or in close proximity to each other on the subject's scalp). The different light detectors may each then be arranged to detect photons of sample light from the same light source which have travelled through similar regions of the subject's brain tissue. The controller 40 may be configured to combine data from the different light detectors (e.g. to average them to provide a single combined read-out for the subject's brain tissue). For example, the controller 40 may be configured to determine cerebral blood flow index data (and optionally also extracerebral data) for the subject's brain tissue based on data obtained using a plurality of optical detectors. This arrangement may yield improved signal to noise for measurements obtained using the iNIRS system 10.

The iNIRS system 10 may include a plurality of source-detector channels which are associated with different regions of the subject's brain tissue. For example, there may be a plurality of optical detectors in different locations on the subject's scalp (these may be coupled to the same light source or different light sources). The controller 40 may be configured to use the plurality of detectors of the iNIRS system 10 to obtain a plurality of ICP measurements (as described above, but for each of a plurality of detectors). The controller 40 may then determine an ICP value for the subject based on the plurality of ICP measurements from the different detectors. This arrangement may increase reliability for measurements, e.g. because ICP may be relatively constant for different regions of the subject's brain tissue.

In examples described above, the iNIRS system 10 may use one source-detector channel to obtain both the cerebral and extracerebral blood flow data. However, the iNIRS system 10 need not obtain the extracerebral blood flow data-for example, an additional sensor may be provided for obtaining extracerebral blood flow pressure values. Additionally, or alternatively, a different iNIRS source-detector channel could be used for obtaining the extracerebral data, such as where the source and detector are arranged closer to each other on the subject's scalp (e.g. to increase selection of shorter travelling sample light photons from source to detector). For example, the iNIRS system 10 may comprise an extracerebral source-detector channel (one source and one detector arranged for measuring extracerebral blood flow, and one or more (e.g. a plurality of) optical detectors arranged for measuring cerebral blood flow.

In examples described above, the extracerebral blood flow data may be obtained using the iNIRS system 10 to measure properties of blood flow in the subject's scalp tissue. However, this should not be considered limiting, as other regions could be used for the extracerebral data. For example, the iNIRS system 10 may be configured to measure properties for the subject's neck, ears, forehead etc., or even for regions further away from their brain tissue (e.g. where more than one source-detector channel is used). The iNIRS system 10 may be configured to obtain blood flow data for a region of the subject's body (not within their skull) where the blood flow being monitored does not have significant external constraints (i.e. where the blood is travelling through a region which is not sufficiently compressed). For example, the extracerebral blood may be flowing in veins/arteries beneath their skin in a region not under great compression by surrounding material.

The iNIRS system 10 may include a plurality of light sources. The light sources may emit light in different wavelength ranges. The iNIRS system 10 may be configured to determine the ICP based on measurements obtained using light in each of a plurality of different wavelength ranges. The iNIRS system 10 may include a first light source configured to emit light in a wavelength range above an oximetry isosbestic wavelength and a second light source configured to emit light in a wavelength range below an oximetry isosbestic wavelength. The controller 40 may be configured to determine ICP based on received light signals from both the first and second light source. The provision of such a first and second light source may enable greater reliability for measurements where blood oxygenation values vary (e.g. because one of the light sources will be emitting light in a wavelength range more suited for absorption by oxygenated haemoglobin, and another light source will be emitting light in a wavelength range more suited for absorption by de-oxygenated haemoglobin.

It will be appreciated in the context of the present disclosure that examples described herein are not intended to be limiting. Instead, examples describe certain potential ways of implementing the claimed technology. For example, the iNIRS system 10 is described with a series of optical cables providing channels and probes for coupling those channels to the subject's scalp. However, it will be appreciated that the probes themselves may be part of the optical channels, or probes may not be provided at all. Similarly, the arrangement of reference channels is just intended to show that reference light is delivered from the light source to the light detectors via optical channels (rather than via the subject's brain tissue). For example, each light source may include one reference channel for each light detector, where that reference channel directly connects the light source to the light detector. In which case, there may be no reference connections in the system at all. Alternatively, and as shown in FIG. 1, the reference light may be transmitted on a common reference optical channel, where some of that reference light is taken from the common reference optical channel to each of the optical detectors. The light source may also be arranged to deliver light to one of a plurality of different locations on the subject's scalp. For example, the light source may be coupled to a plurality of different sample delivery channels, each extended towards the subject's scalp (e.g. from a light splitter).

It will be appreciated that the particular arrangement shown for signal processing circuitry of the detector need not be considered limiting. Each light detecting arrangement 130 is configured to combine sample and reference light to provide a combined light signal with components at one or more beat frequencies, and to process those combined light signals to determine one or more properties of the subject's brain tissue. Any suitable signal processing and/or conversion circuitry could be used for this. For example, a transimpedance amplifier may not be needed (e.g. depending on the photodetector/ADC, no current to voltage conversion may be needed, or this may be performed in a different way). Similarly, a balanced photodetector need not be used, and instead a single photodetector, such as a photodiode, could be used. Similarly, the arrangement with the ADC shown in the Figs. need not be considered limiting. For example, multiple ADCs may be used (e.g. one for each detector output stream), or all detector output streams may be fed into one common ADC.

Additionally, as will be appreciated in the context of the present disclosure, the teaching set out above may be implemented with a Time Domain Diffuse Correlation Spectroscopy (TD-DCS system). For example, an aspect of the present disclosure may provide a TD-DCS system configured to obtain an indication of such pulsatile waveforms for cerebral blood flow, and the controller 40 may be configured to process this TD-DCS data to obtain an indication of ICP.

It will be appreciated from the discussion above that the examples shown in the figures are merely exemplary, and include features which may be generalised, removed or replaced as described herein and as set out in the claims. With reference to the drawings in general, it will be appreciated that schematic functional block diagrams are used to indicate functionality of systems and apparatus described herein. In addition the processing functionality may also be provided by devices which are supported by an electronic device. It will be appreciated however that the functionality need not be divided in this way, and should not be taken to imply any particular structure of hardware other than that described and claimed below. The function of one or more of the elements shown in the drawings may be further subdivided, and/or distributed throughout the apparatus of the disclosure. In some examples the function of one or more elements shown in the drawings may be integrated into a single functional unit.

As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates.

Any controller of the present disclosure may be implemented with fixed logic such as assemblies of logic gates or programmable logic such as software and/or computer program instructions executed by a processor. The controller may comprise a central processing unit (CPU) and associated memory, connected to a graphics processing unit (GPU) and its associated memory. Other kinds of programmable logic include programmable processors, programmable digital logic (e.g., a field programmable gate array (FPGA), a tensor processing unit (TPU), an erasable programmable read only memory (EPROM), an electrically erasable programmable read only memory (EEPROM), an application specific integrated circuit (ASIC), or any other kind of digital logic, software, code, electronic instructions, flash memory, optical disks, CD-ROMs, DVD ROMs, magnetic or optical cards, other types of machine-readable mediums suitable for storing electronic instructions, or any suitable combination thereof. In particular, any controller of the present disclosure may be provided by an ASIC.

Other examples and variations of the disclosure will be apparent to the skilled addressee in the context of the present disclosure.

1. A non-invasive intracranial pressure sensing apparatus comprising an interferometric near infrared spectroscopy, iNIRS, system, the iNIRS system comprising:
a light emitting arrangement comprising:
a light source configured to emit light;
a sample delivery channel coupled to the light source and arranged to be coupled to the subject's scalp to direct light from the light source towards the subject's brain tissue; and
a reference channel coupled to the light source for receiving light therefrom;
a light detecting arrangement configured to be coupled to the subject's scalp and the light emitting arrangement, the light detecting arrangement comprising an interferometric optical detector configured to receive: (i) reference light from the reference channel, and (ii) sample light from the subject's brain, the sample light comprising light emitted from the light source;
wherein the optical detector is arranged to combine the sample light with the reference light to provide combined light signals comprising one or more components at a beat frequency between sample light and reference light; and
wherein the sensing apparatus comprises a controller configured to process data indicative of the combined light signals to determine an indication of intracranial blood pressure for the subject based on at least one property of a pulsatile waveform of one or more identified pulses of blood flow through the subject's brain.

2. The sensing apparatus of claim 1, wherein processing the data indicative of the combined light signals comprises obtaining cerebral blood flow data indicative of one or more pulses of blood flow through the subject's brain; and
wherein the controller is configured to obtain extracerebral blood flow data indicative of one or more pulses of blood flow through an extracerebral region of the subject's body.

3. The sensing apparatus of claim 2, wherein the controller is configured to determine the indication of intracranial blood pressure for the subject based on both the cerebral blood flow data and the extracerebral blood flow data.

4. The sensing apparatus of claim 3, wherein the extracerebral blood flow data contains an indication of blood pressure for the pulses of blood flow through the extracerebral region of the subject's body.

5. The sensing apparatus of claim 4, wherein the controller is configured to determine the indication of intracranial blood pressure for the subject based on: (i) the pulsatile waveform of one or more identified pulses of blood flow through the subject's brain, and (ii) a corresponding pulsatile waveform for the blood pressure for one or more pulses of blood flow through the extracerebral region of the subject's body.

6. The sensing apparatus of claim 5, wherein the controller is configured to determine the indication of intracranial blood pressure for the subject based on an identified critical closing pressure for a blood vessel in the subject's brain.

7. The sensing apparatus of claim 1, wherein the controller is configured to process the data indicative of the combined light signals to obtain cerebral blood flow index data for blood flow through the subject's brain, and
wherein the pulsatile waveform of one or more identified pulses of blood flow through the subject's brain comprises a pulsatile waveform for the cerebral blood flow index.

8. The sensing apparatus of claim 1, wherein the sensing apparatus is configured to obtain the extracerebral blood flow data using the iNIRS system.

9. The sensing apparatus of claim 8, wherein the iNIRS system is configured to obtain extracerebral blood flow index data, and wherein the controller is configured to process the extracerebral blood flow index data to obtain values for extracerebral blood pressure.

10. The sensing apparatus of claim 8, wherein the light detecting arrangement is configured to be coupled to the subject's scalp for the detector to obtain combined light signals comprising both: (i) components at beat frequencies associated with sample light travelling from the subject's brain, and (ii) components at beat frequencies associated with sample light travelling from the extracerebral region of the subject's body.

11. The sensing apparatus of claim 10, wherein the controller is configured to demix data associated with the subject's brain from data associated with the extracerebral region of the subject's body.

12. (canceled)

13. The sensing apparatus of claim 1, wherein the iNIRS system comprises a plurality of optical detectors, wherein each optical detector is configured to obtain cerebral blood flow data for the subject's brain.

14. The sensing apparatus of claim 13, wherein the controller is configured to determine the indication of intracranial blood pressure based on properties of pulsatile waveforms of pulses of blood flow through the subject's brain detected by the plurality of detectors.

15. The sensing apparatus of claim 13,
wherein processing the data indicative of the combined light signals comprises obtaining cerebral blood flow data indicative of one or more pulses of blood flow through the subject's brain;
wherein the controller is configured to obtain extracerebral blood flow data indicative of one or more pulses of blood flow through an extracerebral region of the subject's body; and
wherein the apparatus is configured to obtain extracerebral blood flow data for different extracerebral regions of the subject's body based on combined light signals associated with the different detectors.

16. (canceled)

17. The sensing apparatus of claim 2, wherein the iNIRS system comprises two source-detector channels: (i) a cerebral source-detector channel configured to obtain cerebral blood flow data, and (ii) an extracerebral source-detector channel configured to obtain extracerebral blood flow data.

18. The sensing apparatus of claim 2, wherein the sensing apparatus comprises an extracerebral blood flow sensor configured to obtain extracerebral blood flow data.

19. The sensing apparatus of claim 1, wherein the controller is configured to determine the indication of intracranial blood pressure based on a difference between diastolic and systolic values for the one or more pulses of blood flow through the subject's brain.

20. (canceled)

21. The sensing apparatus of claim 1, wherein the controller is configured to obtain time of flight data based on the combined light signals, wherein the time of flight data comprises a data surface containing a time-ordered series of time of flight distributions for photons of sample light reaching the optical detector from the light source.

22. (canceled)

23. (canceled)

24. A method of non-invasive intracranial pressure sensing, the method comprising:
operating a light source to emit light;
delivering light from the light source through both: (i) a sample channel and towards the subject's brain, and (ii) a reference channel;
receiving at an interferometric optical detector: (i) reference light from the reference channel, and (ii) sample light from the subject's brain, the sample light comprising light emitted from the light source;
combining, at the optical detector, the sample light with the reference light to provide combined light signals comprising one or more components at a beat frequency between sample light and reference light;
processing data indicative of the combined light signals to determine an indication of intracranial blood pressure for the subject based on at least one property of a pulsatile waveform of one or more identified pulses of blood flow through the subject's brain.

25. A non-transitory computer program product comprising computer program instructions configured to program a controller to control operation of a light emitting arrangement and a light detecting arrangement to perform the method of claim 24.
